(19) United States
(12) Patent Application Publication
(10) Pub. No.: US 2026/0166278 A1
(43) Pub. Date: Jun. 18, 2026

(54) WIRE LOCK FOR MEDICAL DEVICES

(71) Applicant: St. Jude Medical, Cardiology Division, Inc., St. Paul, MN (US)

(72) Inventors: John Hong, Prior Lake, MN (US); Troy Tegg, Elk River, MN (US); Manuel Tobias Cajamarca, Shakopee, MN (US); Neil Hawkinson, Ramsey, MN (US); Adam LaVine, Minneapolis, MN (US); Rakshit Shah, Plymouth, MN (US); Tiange Zhang, St. Louis Park, MN (US)

(21) Appl. No.: 19/416,672

(22) Filed: Dec. 11, 2025

Related U.S. Application Data
(60) Provisional application No. 63/733,076, filed on Dec. 12, 2024.

(51) Int. Cl. A61M 25/01

(52) U.S. Cl. CPC A61M 25/0147; A61M 2207/00

(57) ABSTRACT

A pull wire assembly for a steerable medical device includes a wire lock and a wire. The lock includes a core having a frustoconical outer surface and an annular collar including a frustoconical inner surface complementary to the outer surface of the core. The core is inserted into the collar. The wire is inhibited against distal movement by a compressive force arising within the wire lock when the frustoconical surfaces of the core and collar are forced against each other. The wire may be inserted into the lock between the collar and the core, such that it is pinned therebetween. Alternatively, the wire may be inserted through an interior of the core, such that it is pinned between pieces of the core, such as a plurality of tines or two halves of the core.

### CROSS-REFERENCE TO RELATED APPLICATIONS

This application claims the benefit of U.S. provisional application no. 63/733,076, filed 12 Dec. 2024, which is hereby incorporated by reference as though fully set forth herein.

### FIELD

The instant disclosure relates to medical devices, such as catheters and other elongate medical devices. In particular, the instant disclosure relates to a wire lock, as may be employed to secure pull wires in steerable medical devices, such as steerable introducers.

### BACKGROUND

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

Those of ordinary skill in the art will be familiar with steerable medical devices. In such catheters, the orientation and/or configuration of the distal end of the catheter can be controlled by one or more actuators, which are typically located on the catheter's control handle. Although various configurations are known, deflection at the distal end of the medical device is often achieved through the use of a pull wire secured to the deflection mechanism at one end and to the distal end of the catheter (e.g., via a pull ring embedded in the wall of the medical device) at its other end. Manipulation of the actuator places the pull wire in tension, which, in turn, effects changes in the orientation and/or configuration of the distal end of the catheter.

In many extant steerable medical devices, the proximal ends of the pull wires are secured to the deflection mechanism via the use of wire locks. For example, the proximal ends of the pull wires may be soldered to disc-shaped copper wire locks, though the use of solder connections increases the complexity of the manufacturing process.

### BRIEF SUMMARY

Aspects of the instant disclosure relate to wire lock structures that can be used, for instance, to secure pull wires to deflection mechanisms in deflectable catheters and analogous elongate medical devices.

In this regard, disclosed herein is a pull wire assembly for a steerable medical device. The pull wire assembly includes a wire lock and a wire. The wire lock includes: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core, wherein the lock core is inserted into the lock collar. The wire is inserted into the wire lock and inhibited against distal movement by a compressive force arising within the wire lock when the frustoconical outer surface of the distal portion of the lock core is forced against the frustoconical inner surface of the lock collar.

The wire may extend through the wire lock between the lock collar and the lock core, such that the compressive force is exerted on the wire by the frustoconical outer surface of the distal portion of the lock core and the frustoconical inner surface of the lock collar.

The lock core may include a longitudinally-extending exterior groove, wherein at least a portion of the wire is positioned within the groove. A depth of the exterior groove may be less than a thickness of the portion of the wire that is positioned within the groove, such that the wire sits proud of the outer surface of the lock core.

Alternatively, the wire may extend through an interior of the lock core. For instance, the distal portion of the lock core may include a plurality of tines, the wire may extend between the plurality of tines, the compressive force may be exerted on the wire by the plurality of tines.

As another example, the lock core may include a first half and a second half, the wire may extend between the first half and the second half, and the compressive force may be exerted on the wire by the first half and the second half.

As yet another example, the distal portion of the lock core may include a plurality of tines, the lock collar may include a cross member configured to occupy a space between the plurality of tines, the wire may enter a distal end of the wire lock, loop around the cross member, and exit the distal end of the wire lock, and the compressive force may be exerted on the wire by the cross member and the plurality of tines.

It is contemplated for the frustoconical outer surface of the distal portion of the lock core and the frustoconical inner surface of the lock collar to have an angle of between 3 degrees and 10 degrees, such as 7 degrees.

Also disclosed herein is a wire lock including: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; and wherein the wire lock is configured to inhibit a wire inserted into the wire lock against distal movement by a compressive force arising within the wire lock when the frustoconical outer surface of the distal portion of the lock core is forced against the frustoconical inner surface of the lock collar.

The compressive force may be exerted on the wire by the frustoconical outer surface of the distal portion of the lock core and the frustoconical inner surface of the lock collar.

The lock core may include a longitudinally-extending exterior groove configured to receive at least a portion of the wire.

The distal portion of the lock core may include a plurality of tines and the compressive force may be exerted on the wire by the plurality of tines.

The lock core may include a first half and a second half and the compressive force may be exerted on the wire by the first half and the second half.

The distal portion of the lock core may include a plurality of tines, the lock collar may include a cross member configured to occupy a space between the plurality of tines, and the compressive force may be exerted on the wire by the cross member and the plurality of tines.

The frustoconical outer surface of the distal portion of the lock core and the frustoconical inner surface of the lock collar may have an angle of between 3 degrees and 10 degrees, such as 7 degrees.

The instant disclosure also provides a method of manufacturing a steerable medical device including the following steps: securing a distal end of a pull wire to a body of a medical device; routing a proximal end of the pull wire through the body of the medical device to a steering assembly located at a proximal end of the medical device, wherein the steering assembly includes a wire lock; and securing the proximal end of the pull wire to the wire lock. The wire lock includes: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; and wherein the proximal end of the pull wire is inhibited against distal movement by a compressive force arising within the wire lock when the frustoconical outer surface of the distal portion of the lock core is forced against the frustoconical inner surface of the lock collar.

In additional aspects, the instant disclosure relates to a method of modifying a steerable medical device including the steps of: providing an original steerable medical device including a steering assembly operable to deflect the original steerable medical device, the steering assembly including a wire lock that, in turn, includes a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; with the steering assembly further including a wire extending through the wire lock and inhibited against distal movement by a compressive force arising within the wire lock when the frustoconical outer surface of the distal portion of the lock core is forced against the frustoconical inner surface of the lock collar; releasing the compressive force by increasing a separation between the frustoconical outer surface of the distal portion of the lock core and the frustoconical inner surface of the lock collar; adjusting a position of the wire within the wire lock to achieve a preset tension on the pull wire; and reestablishing the compressive force by decreasing the separation between the frustoconical outer surface of the distal portion of the lock core and the frustoconical inner surface of the lock collar.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of an exemplary steerable catheter.

FIG. 2 depicts the handle of the catheter of FIG. 1 with the exterior housing removed in order to visualize certain internal components thereof.

FIG. 3A is a perspective view of a pull wire assembly according to a first aspect of the present disclosure.

FIG. 3B is a cut away side view of the pull wire assembly of FIG. 3A.

FIG. 4A is a perspective view of a pull wire assembly analogous to FIG. 3A, but showing a round pull wire rather than a flat pull wire.

FIG. 4B is a cut away side view of the pull wire assembly of FIG. 4A.

FIG. 5 is an exploded perspective view of a pull wire assembly according to a second aspect of the present disclosure.

FIG. 6 is a perspective view of the pull wire assembly shown in FIG. 5.

FIG. 7 is a side view of a pull wire assembly according to a third aspect of the present disclosure.

FIG. 8 is a cut away side view of the pull wire assembly of FIG. 7.

FIG. 9 is side view of a pull wire assembly according to a fourth aspect of the present disclosure.

FIG. 10 is a perspective view of a pull wire assembly according to a fourth aspect of the present disclosure.

FIG. 11 is a cut away view of the pull wire assembly of FIG. 10.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure relates to wire locks that can be used, for example, to secure pull wires within the deflection mechanism of a deflectable elongate medical device. For the sake of illustration, certain embodiments of the disclosure will be explained herein with reference to a steerable introducer, such as the Agilis™ NxT Steerable Introducer (Abbott Laboratories; Abbott Park, Illinois). It should be understood, however, that the present teachings may be applied to good advantage in other contexts as well (e.g., steerable diagnostic and therapeutic catheters and so forth).

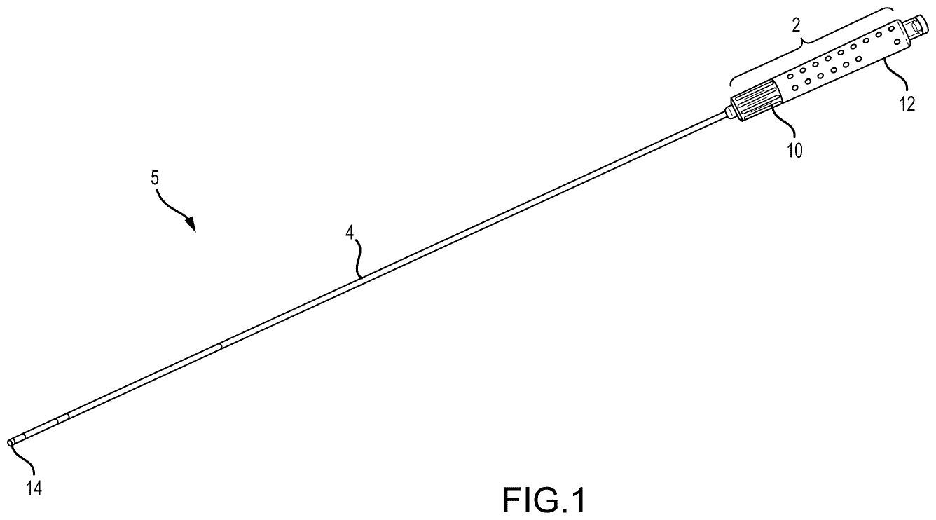

FIG. 1 depicts an exemplary steerable introducer 5. Introducer 5 generally includes a flexible tubular body 4 and a handle 2. It should be understood that the overall length of body 4 as depicted in FIG. 1 is merely exemplary and may vary consistent with the teachings herein, provided, of course, that it is sufficient to reach the intended destination within the patient's body (e.g., the heart).

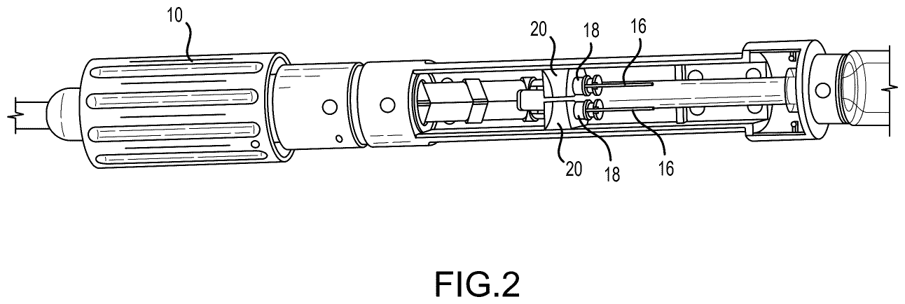

As shown in FIG. 1, handle 2 is connected to body 4 and includes an actuator 10 and a grip portion 12. As those of ordinary skill in the art will appreciate, manipulation of actuator 10 (e.g., rotating actuator 10 in one direction or the other about the longitudinal axis of handle 2) causes distal end 14 of body 4 to deflect (e.g., bi-directionally in a plane) through the action of one or more pull wires 16 (visible, for example, in FIG. 2) extending from within handle 2 to a point within or near distal end 14 of body 4, where they may be secured to body 4 via pull rings or other suitable structures.

In FIG. 2, grip portion 12 of handle 2 has been removed, thus making visible a plurality of pull wires 16 within handle 2. As described in greater detail below, each pull wire 16 is secured to a wire lock 18, which in turn can ride on a respective slider block 20. Thus, as those of ordinary skill in the art will recognize, rotating actuator 10 in one direction or the other will cause the members of a pair of slider blocks 20 to move in opposing directions (that is, one slider block 20 of the pair will move proximally, while the other slider block 20 of the pair will move distally), such as by action of a rack-and-pinion gear system within handle 2. Slider block 20 moving proximally will push wire lock 18 riding thereon proximally, which places corresponding pull wire 16 secured thereto into tension, which, in turn, deflects distal end 14 of body 4.

Pull wires 16 may have various cross-sectional shapes. Those of ordinary skill in the art will be familiar, for example, with the use of both round and flat pull wires 16 in steerable medical devices such as introducer 5.

Further, insofar as the basic construction of steerable catheters will be familiar to those of ordinary skill in the art, it need not be, and will not be, discussed in detail herein, except as necessary to understand the instant disclosure. By way of illustration only, however, U.S. Pat. Nos. 8,734,699, 8,676,290, and 7,691,095 , as well as United States patent application publication no. 2007/0299424, all of which are hereby incorporated by reference as though fully set forth herein, describes various exemplary steerable catheters 5 and/or handles 2 in connection with which the instant teachings may be applied to good advantage.

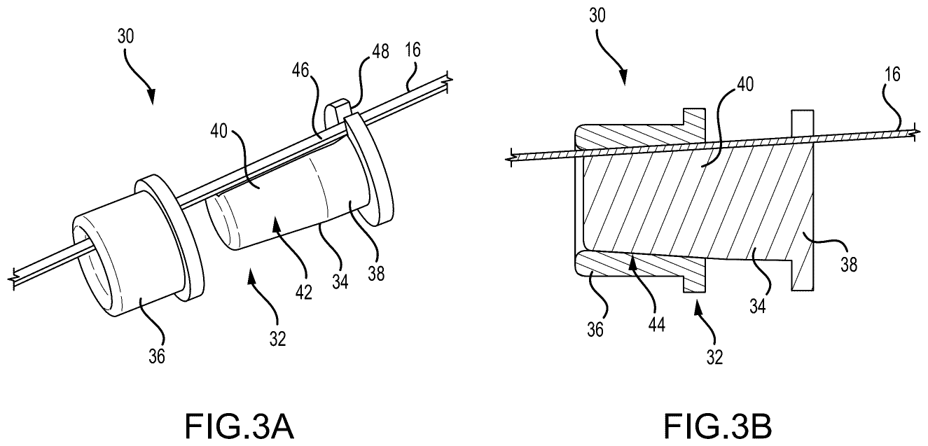

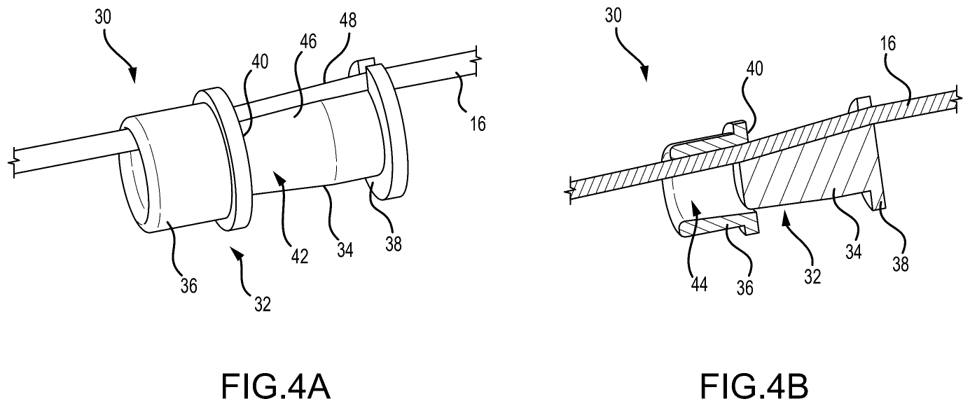

FIGS. 3A, 3B, 4A, and 4B illustrate a pull wire assembly 30 (that is, the connection between a pull wire 16 and a wire lock 32) according to a first aspect of the instant disclosure. As shown in FIGS. 3A, 3B, 4A, and 4B, wire lock 32 generally includes a lock core 34 and an annular lock collar 36. Lock core 34 in turn includes a proximal portion 38 (that is, a portion that is oriented towards the proximal end of handle 2, away from a corresponding slider block 20) and a distal portion 40 (that is, a portion that is oriented towards the distal end of handle 2, towards a corresponding slider block 20).

Lock core 34 and annular lock collar 36 may be cylindrical. As used herein, the term “cylindrical” is not limited to circular and/or oval bases and/or cross sections, as other base and/or cross-sectional shapes are contemplated. Likewise, the term “cylindrical” is not limited to right cylinders. In other words, and as discussed in further detail below, lock core 34 and/or annular lock collar 36 can taper from one end to the other and still be “cylindrical” within the meaning of the instant disclosure.

Distal portion 40 of lock core 34 and annular lock collar 36 include complementary frustoconical surfaces. More particularly, distal portion 40 of lock core 34 includes a frustoconical outer surface 42, while annular lock collar 36 includes a frustoconical inner surface 44. Frustoconical surfaces 42, 44 may have angles of between about 3 degrees and about 10 degrees, such as about 7 degrees.

In use, lock core 34 is inserted into annular lock collar 36 with pull wire 16 (shown as a flat pull wire in FIGS. 3A and 3B and as a round pull wire in FIGS. 4A and 4B) inserted through wire lock 30 between frustoconical outer surface 42 of lock core 34 and frustoconical inner surface 44 of annular lock collar 36 as shown in FIG. 4. More particularly, lock core 34 is inserted into annular lock collar 36 to such an extent that the compressive force acting on pull wire 16 by virtue of being trapped between frustoconical surfaces 42, 44 is sufficient to inhibit distal movement of pull wire 16 when pull wire 16 is pulled distally, as is the case when it is placed in tension through actuation of corresponding actuator 10. In other words, wire lock 30 prevents the tension applied to pull wire 16 by actuation of actuator 10 from extracting or releasing pull wire 16 distally from wire lock 30. In fact, increased tension on pull wire 16 will tend to draw lock core 34 further into annular lock collar 36, which, due to the interaction between frustoconical surfaces 42, 44, will advantageously further increase the compressive force inhibiting distal movement of pull wire 16.

Moreover, because the compressive force acting on pull wire 16 is distributed over a greater length of pull wire 16 than in extant wire locks, the potential for shear failure of pull wire 16 is reduced. For instance, annular lock collar 36 may have an overall length (e.g., along a longitudinal axis running perpendicular to its proximal and distal ends) of between about 2.97 mm (0.117 inches) and about 3.12 mm (0.123 inches). Similarly, lock core 34 may have an overall length (e.g., along a longitudinal axis running perpendicular to its proximal and distal ends) of between about 5.33 mm (0.210 inches) and about 5.49 mm (0.216 inches).

In aspects of the disclosure, lock core 34 may include a longitudinally-extending groove 46 in its exterior surface. Groove 46, which may include a notch 48 in proximal portion 38, accommodates pull wire 16 as it passes from the distal end of wire lock 30 to the proximal end of wire lock 30. Groove 46 may be shallower than the thickness (in the case of a flat pull wire 16) or diameter (in the case of a round pull wire 16) of pull wire 16 to increase engagement between pull wire 16 and frustoconical surfaces 42, 44.

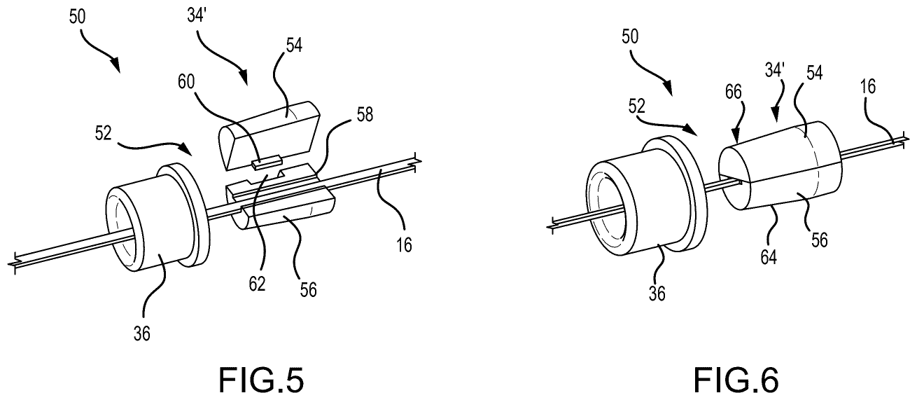

Alternatively, pull wire 16 may extend through an interior of the lock core. For instance, FIGS. 5 and 6 illustrate a pull wire assembly 50 according to a second aspect of the instant disclosure that includes a wire lock 52 having a lock core 34′ and an annular lock collar 36. Pull wire 16 extends along a central longitudinal axis of wire lock 52 (e.g., through lock core 34′).

In wire lock 52, lock core 34′ includes a first half 54 and a second half 56, at least one of which (e.g., second half 56 as illustrated) includes a groove 58 to accommodate pull wire 16. First half 54 and second half 56 also include respective alignment structures (e.g., tab 60 and corresponding slot or cavity 62). Although FIG. 5 illustrates first half 54 entirely disconnected from second half 56, it is contemplated that first half 54 may be hingedly connected to second half 56, allowing lock core 34′ as shown in FIGS. 5 and 6 to open and close in a clamshell manner.

Analogous to distal portion 40 of lock core 34 as described above, distal portion 64 of lock core 34′ includes a frustoconical outer surface 66 that is complementary to frustoconical inner surface 44 of lock collar 36. Suitable angles for frustoconical surfaces 66, 44 are discussed above.

In use, lock core 34′ is inserted into annular lock collar 36, with pull wire 16 inserted through lock core 34′ between first and second halves 54, 56. As lock core 34′ is inserted (or pulled via pull wire 16 in tension) further into annular lock collar 36, the interaction between frustoconical outer surface 66 of lock core 34′ and frustoconical inner surface 44 of lock collar 36 forces first and second halves 54, 56 together with progressively increasing force. This, in turn, progressively increases the compressive force acting on pull wire 16 by virtue of being trapped between first and second halves 54, 56.

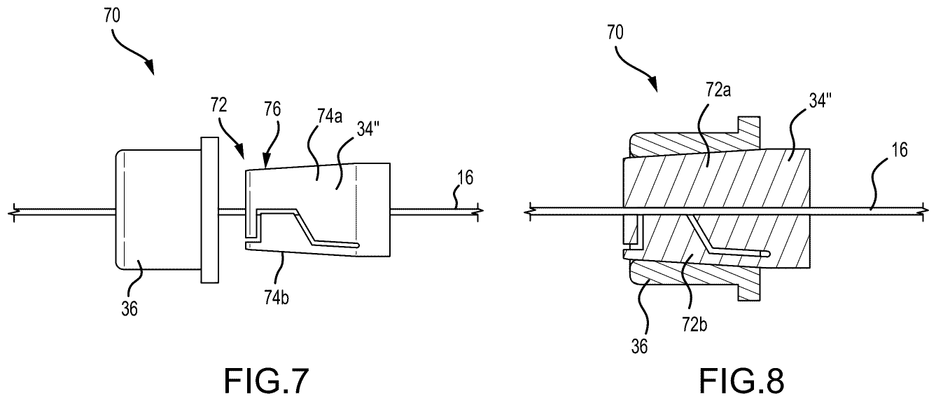

FIGS. 7 and 8 illustrate a pull wire assembly 70 according to yet another aspect of the instant disclosure that includes a wire lock 72 having a lock core 34″ and an annular lock collar 36. Pull wire 16 extends along a central longitudinal axis of lock core 34″. In the interest of brevity, and because certain components, characteristics, and features of pull wire assembly 70 are identical or analogous to those found in pull wire assemblies 30 and/or 50 described above, the following description will highlight certain differences in pull wire assembly 70. One of ordinary skill in the art will appreciate how and the extent to which the description of pull wire assemblies 30 and 50 applies equally to pull wire assembly 70.

In pull wire assembly 70, lock core 34″ includes a plurality of tines 74a, 74b. Although two tines 74a, 74b are shown in FIGS. 7 and 8, this is merely exemplary and lock core 34″ can include any number or configuration of tines without departing from the scope of the present disclosure.

As those of ordinary skill in the art will appreciate from the disclosures herein, as lock core 34″ is inserted into lock collar 36, the interaction of frustoconical inner surface 44 of lock collar 36 and frustoconical outer surface 76 on lock core 34″ will increasingly force tines 74a, 74b together. Thus, the compressive force exerted on pull wire 16 extending through lock core 34″ by tines 74a, 74b will increase as lock core 34″ is inserted (or pulled via pull wire 16 in tension) further into lock collar 36.

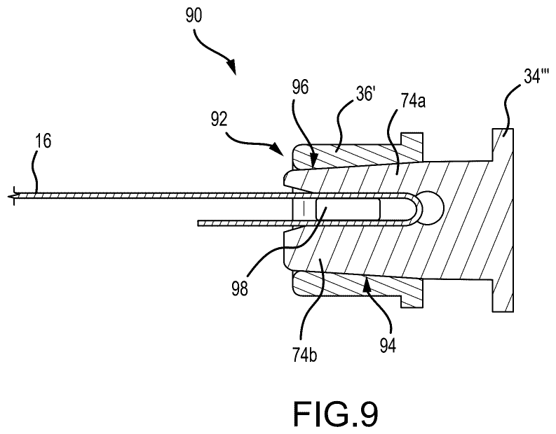

FIG. 9 illustrates a pull wire assembly 90 according to still another aspect of the instant disclosure. To the extent pull wire assembly 90 includes components, characteristics, and features that are identical or analogous to pull wire assemblies 30, 50, and/or 70 discussed above, they will not be described in detail below. Rather, certain differences in pull wire assembly 90 will be highlighted.

Pull wire assembly 90 includes a wire lock 92 having a lock core 34″′ and an annular lock collar 36′. Analogous to lock core 34″, lock core 34″′ includes a plurality of tines 74a, 74b that are driven together by the interaction between frustoconical inner surface 94 of annular lock collar 36′ and frustoconical outer surface 96 of lock core 34″′ as lock core 34″′ is inserted into annular lock collar 36′. Annular lock collar 36′ includes a cross member 98 configured to fit into the space defined between tines 74a, 74b.

In use, pull wire 16 is inserted into the distal end of wire lock 92, looped around the proximal side of cross member 98, and then exits the distal end of wire lock 92. Thus, as lock core 34″′ is inserted (or pulled by pull wire 16 in tension) further into lock collar 36′, the interaction of frustoconical inner surface 94 of lock collar 36′ and frustoconical outer surface 96 on lock core 34″′ will increasingly force tines 74a, 74b against cross member 98, increasing the compressive force exerted on pull wire 16 by tines 74a, 74b, and cross member 98.

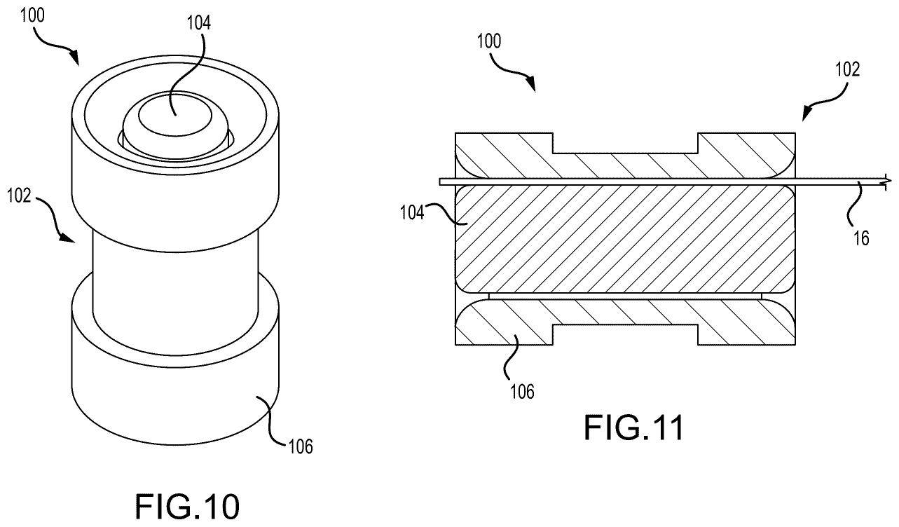

FIGS. 10 and 11 illustrate a pull wire assembly 100 according yet a further aspect of the instant disclosure. Pull wire assembly 100 is well-suited to use with round pull wires. To the extent pull wire assembly 100 includes components, characteristics, and features that are identical or analogous to pull wire assemblies 30, 50, 70 and/or 90 discussed above, they will not be described in detail below. Rather, certain differences in pull wire assembly 100 will be highlighted.

Pull wire assembly 100 includes a wire lock 102 having a lock core 104 and an annular lock collar 106. As visible in FIG. 11, lock core 104 and annular lock collar 106 do not include complementary frustoconical surfaces. Use of wire lock 102 in pull wire assembly 100 is otherwise analogous to pull wire assembly 30.

The use of wire locks as disclosed herein may also be advantageous when reprocessing catheter 5. For instance, over repeated deflections during initial usage of catheter 5, pull wires 16 may become stretched relative to their neutral (that is, original) lengths via plastic deformation, thus creating additional slack in pull wires 16 (e.g., between a wire lock 18 and its corresponding slider block 20) that must be taken up before deflection begins. Over time and repeated deflections, therefore, the deflection range at distal end 14 of catheter 15 may be undesirably diminished as compared to the preset deflection range for which distal end 14 of catheter 5 is originally designed or intended.

During reprocessing, however, the position of a wire lock 18 as disclosed herein along its respective pull wire 16 may be adjusted by releasing wire lock 18 (e.g., by separating lock core 34 and annular lock collar 36), adjusting the tension on pull wire 16 as desired by sliding it relative to now-released wire lock 18, and then re-securing wire lock 18 (e.g., reinserting lock core 34 into annular lock collar 36). Such adjustment can restore most or all of the original deflection range at distal end 14 of catheter 5.

Wire locks as disclosed herein can also be employed to adjust, improve, and/or calibrate the balance between pull wires 16, both during initial manufacture of catheter 5 and during reprocessing of catheter 5. For instance, it is desirable for the amount by which actuator 10 must be displaced to effect a particular amount of deflection at distal end 14 of catheter 5 to be relatively consistent in all directions. This consistency (or “balance”) may not be present, however, if different pull wires 16 have different gaps between their respective slider blocks 20 and wire locks 18 (particularly if the gaps differ with actuator 10 in its neutral position). Accordingly, the position of one or more wire locks 18 along their respective pull wires 16 can be adjusted, during either initial manufacture or reprocessing of catheter 5, in order to improve balance between pull wires 16.

The Following Are Numbered Clauses of the Invention:
Clause 1. A pull wire assembly for a steerable medical device, comprising: a wire lock comprising: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; and a wire inserted into the wire lock and inhibited against distal movement by a compressive force arising within the wire lock when the frustoconical outer surface of the distal portion of the lock core is forced against the frustoconical inner surface of the lock collar.
Clause 2. The pull wire assembly according to clause 1, wherein: the wire extends through the wire lock between the lock collar and the lock core; and the compressive force is exerted on the wire by the frustoconical outer surface of the distal portion of the lock core and the frustoconical inner surface of the lock collar.
Clause 3. The pull wire assembly according to clause 2, wherein the lock core includes a longitudinally-extending exterior groove and wherein at least a portion of the wire is positioned within the groove.
Clause 4. The pull wire assembly according to clause 3, wherein a depth of the exterior groove is less than a thickness of the portion of the wire.
Clause 5. The pull wire assembly according to any preceding clause, wherein the wire extends through an interior of the lock core.
Clause 6. The pull wire assembly according to clause 5, wherein: the distal portion of the lock core includes a plurality of tines; the wire extends between the plurality of tines; and the compressive force is exerted on the wire by the plurality of tines.
Clause 7. The pull wire assembly according to clause 5 or 6, wherein: the lock core comprises a first half and a second half; the wire extends between the first half and the second half; and the compressive force is exerted on the wire by the first half and the second half.
Clause 8. The pull wire assembly according to any of clauses 5-7, wherein: the distal portion of the lock core includes a plurality of tines; the lock collar includes a cross member configured to occupy a space between the plurality of tines; the wire enters a distal end of the wire lock, loops around the cross member, and exits the distal end of the wire lock; and the compressive force is exerted on the wire by the cross member and the plurality of tines.
Clause 9. The pull wire assembly according to any preceding clause, wherein the frustoconical outer surface of the distal portion of the lock core and the frustoconical inner surface of the lock collar has an angle of between 3 degrees and 10 degrees.
Clause 10. The pull wire assembly according to clause 9, wherein the angle is 7 degrees.
Clause 11. A wire lock, comprising: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; and wherein the wire lock is configured to inhibit a wire inserted into the wire lock against distal movement by a compressive force arising within the wire lock when the frustoconical outer surface of the distal portion of the lock core is forced against the frustoconical inner surface of the lock collar.
Clause 12. The wire lock according to clause 11, wherein the compressive force is exerted on the wire by the frustoconical outer surface of the distal portion of the lock core and the frustoconical inner surface of the lock collar.
Clause 13. The wire lock according to clause 12, wherein the lock core includes a longitudinally-extending exterior groove configured to receive at least a portion of the wire.
Clause 14. The wire lock according to any of clauses 11-13, wherein the distal portion of the lock core includes a plurality of tines and the compressive force is exerted on the wire by the plurality of tines.
Clause 15. The wire lock according to any of clauses 11-14, wherein the lock core comprises a first half and a second half and the compressive force is exerted on the wire by the first half and the second half.
Clause 16. The wire lock according to any of clauses 11-15, wherein: the distal portion of the lock core includes a plurality of tines; the lock collar includes a cross member configured to occupy a space between the plurality of tines; and the compressive force is exerted on the wire by the cross member and the plurality of tines.
Clause 17. The wire lock according to any of clauses 11-16, wherein the frustoconical outer surface of the distal portion of the lock core and the frustoconical inner surface of the lock collar has an angle of between 3 degrees and 10 degrees.
Clause 18. The pull wire assembly according to clause 17, wherein the angle is 7 degrees.
Clause 19. A method of manufacturing a steerable medical device, comprising: securing a distal end of a pull wire to a body of a medical device; routing a proximal end of the pull wire through the body of the medical device to a steering assembly located at a proximal end of the medical device, wherein the steering assembly comprises a wire lock; and securing the proximal end of the pull wire to the wire lock, wherein the wire lock comprises: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; and wherein the proximal end of the pull wire is inhibited against distal movement by a compressive force arising within the wire lock when the frustoconical outer surface of the distal portion of the lock core is forced against the frustoconical inner surface of the lock collar.
Clause 20. A method of modifying a steerable medical device, comprising: providing an original steerable medical device including a steering assembly operable to deflect the original steerable medical device, the steering assembly comprising: a wire lock comprising: a lock core having a proximal portion and a distal portion, wherein the distal portion includes a frustoconical outer surface; and an annular lock collar, the lock collar including a frustoconical inner surface complementary to the frustoconical outer surface of the distal portion of the lock core; wherein the lock core is inserted into the lock collar; and a wire extending through the wire lock and inhibited against distal movement by a compressive force arising within the wire lock when the frustoconical outer surface of the distal portion of the lock core is forced against the frustoconical inner surface of the lock collar; releasing the compressive force by increasing a separation between the frustoconical outer surface of the distal portion of the lock core and the frustoconical inner surface of the lock collar; adjusting a position of the wire within the wire lock to achieve a preset tension on the pull wire; and reestablishing the compressive force by decreasing the separation between the frustoconical outer surface of the distal portion of the lock core and the frustoconical inner surface of the lock collar.
Clause 21. A wire lock, comprising: a cylindrical lock core having an outer surface; and an annular lock collar having an inner surface complementary to the outer surface of the lock core; wherein the lock core is inserted into the lock collar; and wherein the wire lock is configured to inhibit a round wire inserted into the wire lock against distal movement by a compressive force arising within the wire lock when the outer surface of the lock core is forced against the inner surface of the lock collar.

Although several embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A pull wire assembly for a steerable medical device, comprising:
a wire lock comprising:
a lock core having a proximal portion and a distal portion, wherein the distal portion includes a frustoconical outer surface; and
an annular lock collar, the lock collar including a frustoconical inner surface complementary to the frustoconical outer surface of the distal portion of the lock core;
wherein the lock core is inserted into the lock collar; and
a wire inserted into the wire lock and inhibited against distal movement by a compressive force arising within the wire lock when the frustoconical outer surface of the distal portion of the lock core is forced against the frustoconical inner surface of the lock collar.

2. The pull wire assembly according to claim 1, wherein:
the wire extends through the wire lock between the lock collar and the lock core; and
the compressive force is exerted on the wire by the frustoconical outer surface of the distal portion of the lock core and the frustoconical inner surface of the lock collar.

3. The pull wire assembly according to claim 2, wherein the lock core includes a longitudinally-extending exterior groove and wherein at least a portion of the wire is positioned within the groove.

4. The pull wire assembly according to claim 3, wherein a depth of the exterior groove is less than a thickness of the portion of the wire.

5. The pull wire assembly according to claim 1, wherein the wire extends through an interior of the lock core.

6. The pull wire assembly according to claim 5, wherein:
the distal portion of the lock core includes a plurality of tines;
the wire extends between the plurality of tines; and
the compressive force is exerted on the wire by the plurality of tines.

7. The pull wire assembly according to claim 5, wherein:
the lock core comprises a first half and a second half;
the wire extends between the first half and the second half; and
the compressive force is exerted on the wire by the first half and the second half.

8. The pull wire assembly according to claim 5, wherein:
the distal portion of the lock core includes a plurality of tines;
the lock collar includes a cross member configured to occupy a space between the plurality of tines;
the wire enters a distal end of the wire lock, loops around the cross member, and exits the distal end of the wire lock; and
the compressive force is exerted on the wire by the cross member and the plurality of tines.

9. The pull wire assembly according to claim 1, wherein the frustoconical outer surface of the distal portion of the lock core and the frustoconical inner surface of the lock collar has an angle of between 3 degrees and 10 degrees.

10. The pull wire assembly according to claim 9, wherein the angle is 7 degrees.

11. A wire lock, comprising:
a lock core having a proximal portion and a distal portion, wherein the distal portion includes a frustoconical outer surface; and
an annular lock collar, the lock collar including a frustoconical inner surface complementary to the frustoconical outer surface of the distal portion of the lock core;
wherein the lock core is inserted into the lock collar; and
wherein the wire lock is configured to inhibit a wire inserted into the wire lock against distal movement by a compressive force arising within the wire lock when the frustoconical outer surface of the distal portion of the lock core is forced against the frustoconical inner surface of the lock collar.

12. The wire lock according to claim 11, wherein the compressive force is exerted on the wire by the frustoconical outer surface of the distal portion of the lock core and the frustoconical inner surface of the lock collar.

13. The wire lock according to claim 12, wherein the lock core includes a longitudinally-extending exterior groove configured to receive at least a portion of the wire.

14. The wire lock according to claim 11, wherein the distal portion of the lock core includes a plurality of tines and the compressive force is exerted on the wire by the plurality of tines.

15. The wire lock according to claim 11, wherein the lock core comprises a first half and a second half and the compressive force is exerted on the wire by the first half and the second half.

16. The wire lock according to claim 11, wherein:
the distal portion of the lock core includes a plurality of tines;
the lock collar includes a cross member configured to occupy a space between the plurality of tines; and
the compressive force is exerted on the wire by the cross member and the plurality of tines.

17. The wire lock according to claim 11, wherein the frustoconical outer surface of the distal portion of the lock core and the frustoconical inner surface of the lock collar has an angle of between 3 degrees and 10 degrees.

18. The pull wire assembly according to claim 17, wherein the angle is 7 degrees.

19. A method of manufacturing a steerable medical device, comprising:
securing a distal end of a pull wire to a body of a medical device;
routing a proximal end of the pull wire through the body of the medical device to a steering assembly located at a proximal end of the medical device, wherein the steering assembly comprises a wire lock; and
securing the proximal end of the pull wire to the wire lock,
wherein the wire lock comprises:
a lock core having a proximal portion and a distal portion, wherein the distal portion includes a frustoconical outer surface; and
an annular lock collar, the lock collar including a frustoconical inner surface complementary to the frustoconical outer surface of the distal portion of the lock core;
wherein the lock core is inserted into the lock collar; and
wherein the proximal end of the pull wire is inhibited against distal movement by a compressive force arising within the wire lock when the frustoconical outer surface of the distal portion of the lock core is forced against the frustoconical inner surface of the lock collar.

20. A method of modifying a steerable medical device, comprising:
providing an original steerable medical device including a steering assembly operable to deflect the original steerable medical device, the steering assembly comprising:
a wire lock comprising:
a lock core having a proximal portion and a distal portion, wherein the distal portion includes a frustoconical outer surface; and
an annular lock collar, the lock collar including a frustoconical inner surface complementary to the frustoconical outer surface of the distal portion of the lock core;
wherein the lock core is inserted into the lock collar; and
a wire extending through the wire lock and inhibited against distal movement by a compressive force arising within the wire lock when the frustoconical outer surface of the distal portion of the lock core is forced against the frustoconical inner surface of the lock collar;
releasing the compressive force by increasing a separation between the frustoconical outer surface of the distal portion of the lock core and the frustoconical inner surface of the lock collar;
adjusting a position of the wire within the wire lock to achieve a preset tension on the pull wire; and
reestablishing the compressive force by decreasing the separation between the frustoconical outer surface of the distal portion of the lock core and the frustoconical inner surface of the lock collar.

21. A wire lock, comprising:
a cylindrical lock core having an outer surface; and
an annular lock collar having an inner surface complementary to the outer surface of the lock core;
wherein the lock core is inserted into the lock collar; and
wherein the wire lock is configured to inhibit a round wire inserted into the wire lock against distal movement by a compressive force arising within the wire lock when the outer surface of the lock core is forced against the inner surface of the lock collar.
